(19) United States
(12) Patent Application Publication
(10) Pub. No.: US 2017/0050052 A1
(43) Pub. Date: Feb. 23, 2017

(54) PATIENT-SPECIFIC, MULTI-MATERIAL, MULTI-DIMENSIONAL ANTHROPOMORPHIC HUMAN EQUIVALENT PHANTOM AND HARDWARE FABRICATION METHOD

(71) Applicants: Eric A. Burgett, Pocatello, IA (US); Rebecca Howell, Houston, TX (US)

(72) Inventors: Eric A. Burgett, Pocatello, IA (US); Rebecca Howell, Houston, TX (US)

(21) Appl. No.: 15/205,800

(22) Filed: Jul. 8, 2016

Related U.S. Application Data
(60) Provisional application No. 62/190,444, filed on Jul. 9, 2015.

(51) Int. Cl. A61N 5/10; B33Y 30/00; B29C 67/00; A61B 6/00

(52) U.S. Cl. CPC A61N 5/1071; A61N 5/1039; A61B 6/583; B33Y 30/00; B29C 67/0055

(57) ABSTRACT

The present invention relates generally to a system and method for improving the quality assurance/quality control (QA/QC) of advanced radiation treatment techniques using a patient-specific, multi-material, multi-dimensional anthropomorphic human equivalent phantom technology.

### CROSS-REFERENCE TO RELATED APPLICATIONS

The present application claims priority to and the benefit of the filing date of U.S. Provisional Patent Application Serial No. 62/190,444, filed Jul. 9, 2015, which is incorporated herein by reference,

### BACKGROUND OF THE INVENTION

The present invention relates generally to a system and method for improving the quality assurance/quality control (“QA/QC”) of advanced radiation treatment techniques using a patient-specific, multi-material, multi-dimensional anthropomorphic human equivalent phantom technology.

Radiation therapy of cancer has evolved significantly over the last few decades. Advances in treatments have strived to minimize the radiation dose delivered to the healthy tissue surrounding the active cancer volume while maximizing the efficacy of the dose delivered to the actual cancer. This has been accomplished through advances in high resolution imaging, such as Computed Tomography (“CT”), Magnetic Resonance Imaging (“MRI”), and Ultrasound; advanced radiation treatment techniques that increase the number of treatment fields, such as altering the directions in which the radiation is externally applied, intensity-modulated radiation therapy, volumetric modulated arc therapy; and treatment techniques that time-gate the application of the radiation, such as real-time tumor tracking radiotherapy and respiration gated radiation therapy.

With the increase in complexity of radiation treatments, more sophisticated. QA/QC techniques are needed to ensure effective treatment while maintaining patient safety. In many instances, existing QA/QC techniques are not adequate for these advanced treatment techniques and new techniques are required. A patient-specific, multi-material, multi-dimensional anthropomorphic human equivalent phantom has been created that uses tissue and bone equivalent materials that will improve the efficacy and safety of modern radiation therapy.

Historically, phantoms, also known as surrogates, for humans have been used to calibrate instrumentation and quantify radiation dose delivered to patients. Phantoms are used during the commissioning of accelerators used for radiation therapy, as well as to obtain daily, weekly, monthly, quarterly, and yearly validation and verification measurements. Typically, they have been constructed of tanks, either rectangular or cubic, containing water or other liquid intended to mimic soft tissue. Water-filled tanks are the most common today. Alternatively, stacks of polymer-based materials under various trade names, such as virtual water or solid water, are used as a simpler and more easy to use phantom system. Today, a user can purchase anthropomorphic phantoms, which have materials that more closely mimic the behavior of the tissue of their human counterparts, but these phantoms are not exact replicas for the patient themselves. All of these currently-available phantom systems were designed to verify radiation beam uniformity as well as the spatial accuracy of the dose delivered prior to patient treatment. Such systems were excellent phantoms for their time. However, with the introduction of more sophisticated treatment techniques, such as intensity-modulated radiation therapy (“IMRT”) and dynamic continuous arc therapy, these traditional QA/QC tools and techniques are inadequate and result in margins of error that are larger than necessary, thereby increasing the chances that a patient will suffer secondary cancers or other damage to otherwise healthy tissues.

With the increase in complexity of radiation treatments, more sophisticated QA/QC techniques are needed to ensure effective treatment while maintaining patient safety.

The phantom system and method described herein uses tissue and bone equivalent materials that will improve the efficacy and safety of modern radiation therapy.

### BRIEF SUMMARY OF THE INVENTION

Among the several objects of this invention may be noted the provision of a system and method for, as described and shown herein, a patient-specific, anthropomorphic phantom that closely mimics patient size and other important radiation characteristics, such as attenuation, absorption, scattering, and pair production, because it is fabricated directly from the patient's diagnostic images, including Computed Tomography (“CT”), Magnetic Resonance Imaging (“MRI)”, and Ultrasound, using additive manufacturing techniques, also known as “3D printing.”

A patient-specific, anthropomorphic phantom made using advanced polymers and fabrication techniques that create tissue and bone-equivalent materials that allow the phantom to accurately model a specific patient's human tissue or bone density and energy-dependent effective Z numbers. The anthropomorphic phantom can be rigid (three-dimensional or “3D”) or capable of time-varying deformations (four-dimensional or “4D”) that model respiration, circulation and digestion. 4D deformable phantoms may be comprised of deformable materials. In one example, for digestive systems, the deformable materials of the digestive tract can be manipulated, by being filled or voided, with surrogate materials that mimic real patient conditions. In another example, for respiratory and pulmonary systems, the lungs, bronchi, and associated tissues are printed as empty voids which are then inflated using an external, high-pressure, controlled and regulated system to mimic a patient's real life respiration. In yet another example, for cardiac systems, the fluidic pressure is regulated with a suitable pump type and the patient heart is mimicked through electromechanical system including but not limited to servo, actuator, piezoelectric, and electromechanical systems.

The advanced polymers can be made of transparent or nearly transparent photo- or radio-chromic polymers that change color when exposed to ionizing or non-ionizing radiation (“direct-reading”) or made with opaque materials implanted with miniature dosimeters (“indirect-reading”) that, like the phantom itself, are made from polymers that accurately model tissue/bone density and energy-dependent effective Z numbers so as to make them non-field perturbing and thus minimize any inaccuracies due to instrumentation in the phantom.

Miniature, non-field perturbing radiation dosimeters made of tissue and/or bone-equivalent materials can be utilized that have effectively no influence on the measured radiation field compared to current measurement techniques and can be precisely placed in the specific locations of the phantom that requires measurement.

A specially-defined registration process is used that allows a patient's medical diagnostic images to be automatically and directly translated into instructions for the 3D printer that fabricates the patient-specific phantom.

A 3D printer is also used to fabricate a patient-specific, anthropomorphic phantom directly from the patient's diagnostic images.

With the direct-reading phantom, the image of the radiation field can be superimposed onto the patient's medical images and provide a very accurate understanding of the radiation field in that patient for a given treatment plan. With the indirect-reading phantom, data points provided by each of the individual miniature dosimeters can be used to create a realistic dose map that can then be superimposed onto the patient's medical images and provide a very accurate understanding of the radiation field in that patient for a given treatment plan.

This invention allows each phantom to be patient-specific in that it is a direct conversion of the patient's medical images into a full-size replica made from tissue- and bone-equivalent material. For the first time, treatment centers will have the ability to effectively and confidently link a patient's treatment plan's planned dose to the dose delivered to the patient.

### BRIEF DESCRIPTION OF THE DRAWINGS

FIG. 1 is a diagram representing an exemplary system for creation of non-field perturbing dosimeters.

FIG. 2 is a diagram representing an exemplary module 3D printing system.

FIGS. 2A and 2B are partial views of the diagram, shown in FIG. 2, representing an exemplary module 3D printing system extended over two sheets.

FIG. 3 is a flow chart showing the steps of a specially-defined registration process in accordance with a preferred embodiment of the present invention.

### DETAILED DESCRIPTION OF THE INVENTION

### The 3D Direct-Reading Phantom

In accordance with one aspect of the invention, the 3D direct-reading phantom, is a non-time-dependent phantom where the phantom itself serves as a radiation dose measurement device. Here, a transparent polymer, which hardens to form a rigid phantom, is used as the matrix and binder for a photochromic/radio-chromic polymer compound. The transparent polymer matrix and binder is fabricated from tissue and bone-equivalent materials that allow the phantom to accurately model a specific patient's tissue/bone density and energy-dependent effective Z numbers such that the phantom is dimensionally accurate within 100 μm of the patient. The photochromic/radio-chromic compound bound onto the binder/matrix reacts with radiation directed onto the phantom. This can be ionizing radiation or non-ionizing, e.g. visible or ultraviolet light depending on the photo-/radio-chromic polymer compound used.

Upon irradiation, the polymer—transparent matrix bound with bound photo-, radio-chromic compound—transitions from transparent at most optical wavelengths to selectively absorbing light in a specific wavelength, i.e. it changes color. This change in optical light absorption (i.e., color) is proportional to the radiation dose delivered; the absorbed wavelengths shift proportionally to the amount of radiation absorbed by the material. This color change provides an almost infinite resolution to the spatial data of the radiation dose delivered.

Then, using conventional CT techniques, the phantom can be scanned either with ambient light or raster-scanned with a specific wavelength of light to quantify the absorbed dose at each point in the phantom thereby providing a very accurate image/map of the delivered dose to the phantom. This image of the radiation field can be superimposed back onto the patient's medical images and provide a very accurate understanding of the radiation field in that patient for a given treatment plan. To minimize imaging aberrations, the phantom may be immersed in a refractive index matching fluid to minimize scattering. Imaging is then undertaken in the fluid and irradiation dose response data is then obtained.

The 3D direct-reading phantom is printed using high Shore Hardness polymers as a matrix that is then doped with photo- or radio-chromatic polymers, including multi-part resins and/or elastomers such as diarylethenes, azobenzenes, phenoxynaphthacene quinone. Polymer selection suitable for the matrix of the direct-reading phantom must be transparent matrix, and include thermoset polymers, multi-part resins, vinyls, urethanes, and elastomers which are binary, ternary, or multi-part, including a resin base and a hardener and can be polymers of acrylates, ethylenes, esters, and the like, e.g., acrylonitrile butadiene styrene (ABS), polylactic acid (PLA), polyvinyl alcohol (PVA), polymethyl methacrylate (PMMA), high-density polyethylene (HDPE), polyethylene (PE), low-density polyethylene (LDPE), and fluorinated and chlorinated plastics like polyvinyl chloride (PVC), polytetrafluoroethylene (PTFE), polystyrenes, and others. The materials suggested herein are specific to tissue, bone, and other materials in which the opacity or transmission of optical light through the polymers is proportional to the amount of absorbed radiation which has been delivered, for example, ionizing or non-ionizing depending on whether radio-chromic or photo-chromic materials used.

The ability to mimic important properties such as densities and energy dependent effective Z numbers of many classes of tissue and bone, e.g. fats, muscles, cortical bones, spongy bone, is accomplished through enhanced technique of cross-linking using techniques including e-beam, ion beam, photon (X-ray), and ultraviolet curing/cross linking techniques which can be applied during the fabrication process or prior to 3D printing during the extrusion process; the additions of various materials including elemental species; and other blends of plastic compounds listed above.

Tissue densities are controlled through the addition of air, water, solvents, ethylene, and others, to achieve the correct densities and energy dependent effective Z numbers above as part of the fabrication process.

Bone densities are controlled through the addition of calcium, strontium, silicon, aluminum, potassium, and other bone trace elements and metals usually in an oxide or calcined form to achieve the correct densities and energy dependent effective Z numbers above as part of the fabrication process.

The major modifier for density is water, solvents and air. The major modifier for z-effective is the addition of elemental and molecular species which get homogenized and integrated into the plastics including calcium compounds, sodium compounds, and other transition metal based compounds depending on the tissue, bone, or other bodily material being printed. Cross linking modifies primarily the adhesion and stability of the plastics, modifying its thermoset point, its rigidity, its radiation tolerance, such as not breaking down or falling out of solution.

The photo- or radio-chromic polymers added to the matrix can be diarylethenes, azobenzenes, or phenoxynaphthacene quinone, as well as metal halides including but not limited to zinc halides and silver halides that may change color proportionally to the absorbed ionizing radiation dose to the tissue equivalent materials. These photo- or radio-chromic materials are mixed into the transparent polymer matrix described above as part of the fabrication process. Photo- or radio-chromic polymers by means of doping with diarylethenes, azobenzenes, and phenoxynaphthacene quinone will transition back to transparent after a decay period allowing for reuse.

After fabrication is completed, the phantom is transparent, or nearly so. Following irradiation, the radio-chromic/photochromic materials darken and change color proportionally to the absorbed radiation dose. The phantom is then digitally imaged in a medium such as in air or in another fluid with an index of refraction matching the phantom to record the colors within the phantom. This digitized image data can then be overlaid on an image of the patient's anatomy to create a composite image that maps dose received during the treatment to specific locations in the patient's treatment area. This technique can provide 100% imaging coverage of the cancer area and surrounding tissues enhancing understanding of delivered dose. Imaging and quantification are accomplished through optical systems such as digital optical scanning and optical filtering or through directed laser scanning of the peak wavelengths of absorption. Three dimensional scanning is completed and reconstructed using standard commercially-available computer software.

### The 3D Indirect-Reading Dosimeter Phantom

A second instantiation of the invention, the 3D indirect-reading dosimeter phantom is fabricated using high Shore Hardness polymers as a matrix that can be transparent or opaque, and is not, itself, a radiation dose measurement device. Instead, non-field perturbing radiation dosimeters (as described below) are added as part of the fabrication process.

After fabrication is completed, the phantom is transparent, or nearly so or opaque with electrical leads from each of the implanted dosimeters available at the surface of the phantom for connection to monitoring instrumentation. During irradiation, the non-field perturbing radiation dosimeters made of tissue and/or bone-equivalent materials are monitored with their signal recorded for later analysis. Data recorded from each implanted dosimeter provides data on delivered dose at that point in the phantom over time. The data from all the dosimeters in the phantom can then be used to create a realistic dose map that can then be superimposed onto the patient's medical images and provide a very accurate understanding of the radiation field in that patient for a given treatment plan.

In all other respects, the 3D dosimeter phantom is identical to the 3D direct-reading phantom.

### The 4D Direct Reading Phantom

A third instantiation of the invention is the 4D direct-reading phantom. With the advancement of the capabilities of time-gated, intensity-modulated radiation therapy, an improved QA/QC system is needed to verify the time-dependent delivery on a patient-specific basis. The 4D capability (time-dimensional dependence) comes from modifying the material formulation of the tissue-equivalent materials, such as through the attachment of the phantom to a mechanical means. The mechanical means include but not limited to a mechanical respirator (breathing), a mechanical pump (artificial heart), peristaltic, piston, or other pump (digestive system). As opposed to obtaining transparent, tissue-equivalent materials from high-rigidity polymers with high Shore Hardness, low Shore Hardness material formulations, e.g. ethylene propylene diene monomer (“EDPM”), silicone rubbers, and others, are used to create the 4D phantom. These soft transparent polymers that mimic a patient's tissue/bone density and energy-dependent effective Z numbers are alloyed with photo-/radio-chromic polymer compounds that change colors during irradiation as described above. This provides radiation dose response with patient-specific tissue morphologies as a function of time.

Polymer formulations for the 4D dosimeter phantom include multi-part resins and/or elastomers which are binary, ternary, or multi-part including a resin base and a hardener. Tissue densities are controlled through the addition of air, water, solvents, ethylene, others to achieve the correct densities and energy dependent effective Z numbers. In this instantiation of the invention, the elastic properties of the polymer material(s) are tuned to more closely represent that of the patient's tissues and bones. The elastic properties are controlled through the precise control of chemicals used in the formulation as well as a controlled degree of cross-linking and cross-linking techniques including e-beam, ion beam, photon (X-ray), and Ultraviolet (“UV”) curing/cross linking techniques which can be applied during the fabrication process or prior to printing during the extrusion process.

During the fabrication process, internal organs that normally move within the patient over time are rendered hollow, e.g., heart, lungs, stomach, colon, intestines, and then controlled externally to mimic their behavior. For example, digestive organs can be filled or voided with air and/or water or other materials depending on specific patient conditions. Respiratory function can be simulated by attaching a respirator to inflate/deflate the lung volumes with air to simulate the body's natural movement due to breathing. Cardiac function can be simulated through attaching an external mechanism to control the motions of the heart. In these ways, time-dependent physical and deformable characteristics and motions of the body can be accounted for and used to obtain more accurate measurement of delivered doses during advanced treatment techniques.

In all other respects, the 4D direct-reading phantom is identical to the 3D direct-reading phantom described herein.

### The 4D Indirect-Reading Dosimeter Phantom

Another instantiation of the invention is the 4D indirect-reading phantom, with non-field disturbing dosimeters as described below embedded in the phantom during its fabrication.

In all other respects, the 4D dosimeter phantom is identical to the 4D direct-reading phantom described above.

### Non-Field Disturbing Dosimeters

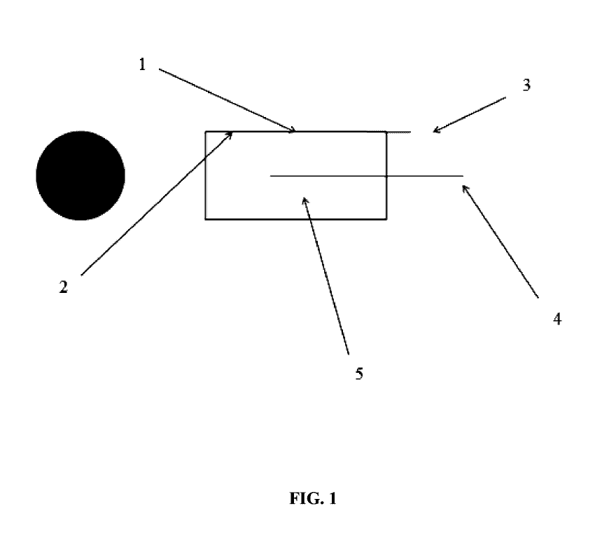

As depicted in FIG. 1, the non-field perturbing dosimeters are created using the same tissue or bone equivalent matrix polymers described above. In one instantiation, a chamber wall 1 is made from bone and/or tissue equivalent material. The materials used to make the chamber wall 1 and central terminal can be constructed of from tissue equivalent materials that are made conducting through the inclusion of metallic or carbon materials. The chambers may also include a set of one or more coaxial cables to apply a voltage potential to the dosimeter and to provide a readout of dosimetry data. The coaxial cables may also be connected to electrometer or precision capacitor which can be multiplexed to read out numerous dosimeters simultaneously.

Such dosimeters may be a solid-state material comprising a semiconductor diode dosimeter operating in pulse or current modes, a solid-state crystalline scintillator which can be fiber optically coupled to a readout device, and a solid-state crystalline, amorphous, or powdered material which stores absorbed ionizing radiation in its crystalline lattice and can be read out after irradiation such as an Optically Stimulated Luminescence (“OSL”) dosimeter or Thermos-Luminescent Dosimeter (“TLD”).

Another instantiation of these dosimeters is of a direct-reading nature comprising polymers cast into a transparent matrix such as PMMA including multi-part resins and/or elastomers which are binary, ternary, or multi-part including a resin base and a hardener to which chemicals are added to the formulation such as diarylethenes, azobenzenes, and phenoxynaphthacene quinone, as well as metal halides including but not limited to zinc halides and silver halides that change color proportionally to the absorbed ionizing radiation dose to the tissue equivalent materials. In this instantiation the dosimeters would have to be read after the dose is received and would not provide real-time dose information.

As shown on FIG. 1, the interior of the dosimeter's ion chamber 2 is made conductive through the addition of powdered carbon or conductive-carbon composites, such as colloidal metallic or metallic conductive paint. Alternatively, a very thin metallic layer can be flash evaporated or deposited onto the dosimeter chamber's interior wall. The anode 3 and cathode 4 of these miniature dosimeters are electrically connected to the exterior of the phantom for direct-reading either through interconnect wires or through wire-bonded or printed circuit traces made with conductive polymers deposited in the phantom layers themselves during the 3D printing process. Functioning as a small, gas-filled ionization chamber 5 (<0.1 cubic centimeter), radiation-induced ionization events produce ion-electron pairs in the chamber. A small applied voltage between the anode and cathode allows the ion-electron pairs to be collected on the anode and cathode. This collected charge is then read out on a micro-, nano-, or pico-ammeter depending on the amount of charge collected. The charge collected is proportional to the dose delivered and responds linearly with increases in total dose and dose rate.

These non-field perturbing gas-filled ionization chambers acting as miniature dosimeters can be inserted or printed directly into the phantom at precise locations required to measure treatment doses.

### Fabrication of the Phantom

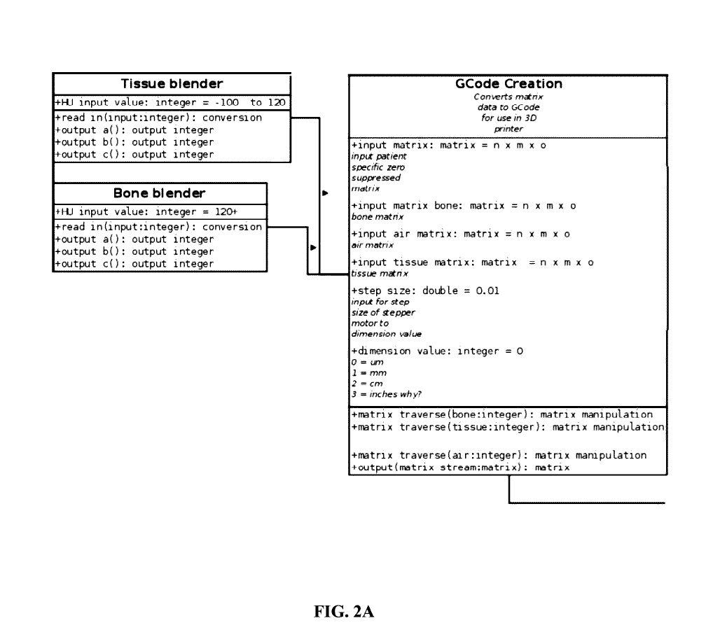

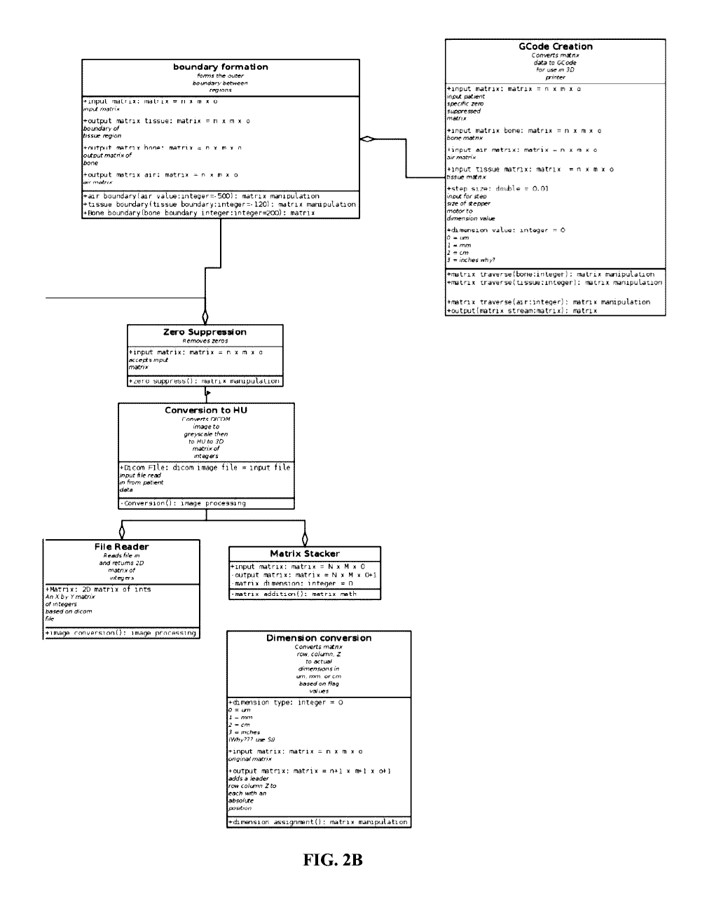

The phantom is created from a patient's medical images using a 3D printing process, also known as fused-deposition modeling (“FDM”). Medical imaging data that can be used as the basis for the phantom include Magnetic Resonance imaging (“MRI”). Computed Tomography (“CT”), ultrasound, endoscopy, or interrogation techniques where three- and/or four-dimensional data of the patient is obtained. Currently, these images are acquired as a series of two-dimensional slices which are stacked in both the third and fourth dimensions. This data is typically saved in a Digital imaging and Communications in Medicine (“DICOM”) format and is then digitized into voxel formats. The data usually consists of greyscale digital images made up of pixels of linear attenuation coefficient measurements translated into Hounsfield Units (“HU”). The HU scale is a linear transformation of the original linear attenuation coefficient measurement into one in which the radio-density of distilled water at standard pressure and temperature (“STP”) is defined as zero HU and the radio-density of air at STP is defined as 1000 HU. These stacked digitized images are used to create a three- or four-dimensional matrix of density values. An specially-defined registration process, shown on FIG. 2, has been created to translate this medical imaging data into instructions for use by the 3D printer that will fabricate the patient-specific phantom.

For the simplest distillation of this technique to practice, three digital HU values are selected, a tissue value, an air value, and a bone value. A 3D model is defined from these values. The model is then tessellated (conversion from 3D cubic format to a nodal mesh of triangles) using commercially-available software to smooth out the non-uniformities in the mesh. This creates a patient-specific, position-specific spatially dependent map of the patient correlating X, Y, Z and time coordinates of the patient to the coordinate system used in printing.

The tessellated surface is then further smoothed to minimize the angular variation as a function of position of the planar surfaces with their adjoining surfaces until a smoothed object is obtained from the pixelated rectangular data Obtained from the medical image. The 3D model and the density-value matrix data are then converted into a computer language capable of operating the 3D printer that is described below. One of two printing techniques is used for construction of the phantom; a direct-printed, fused-deposition technique or a spatially-dependent, direct-printing technique.

The direct-printed FDM technique uses a 3D printer to create the three component phantom type which bonds tissue-equivalent or bone-equivalent materials to the layer of materials previously laid down by the printer. A simple phantom is made that uses one head of a dual-head 3D printer to print the tissue-equivalent material while the second head prints bone-equivalent material. In this way a phantom with air spaces and voids, tissues, and bones, is accurately rendered that corresponds directly to the patient model created using the specially-defined registration process. Inaccuracies will exist in the use of a single tissue-equivalent material to model all tissues in the phantom. However, this is still more accurate than current techniques. This method could be used to create a bone surrogate that mimics the density, radiation attenuation, absorption, and scattering, and pair production properties of a patient's tissues or a tissue surrogate that mimics the density, radiation attenuation, absorption, and scattering, pair production properties of tissue.

The second alternative printing technique for 3D printing a three component phantom (i.e., air spaces/voids, tissues, and bones) acknowledges the difficulties of printing a realistic bone material. The bone material requires a significantly higher temperature and can experience delamination from layer to layer. To overcome this, a multipart polymer compound is made in bulk liquid form and the 3D printer is used to create a hollow shell fully encompassing the boundary of the tissue volume. This is to assist in higher density bone creation which can be a challenge to 3D print. An infill may or may not be used to support the structure which is being printed which may or may not be removed, dissolved, or otherwise subtracted from the final product.

The shell structure is then filled with tissue-equivalent material and/or bone-equivalent material. These materials are described in the following sections. As part of the fabrication process, miniature radiation dosimeters can be printed using tissue- and/or bone-equivalent materials to provide a non-field perturbing radiation detection and measuring capability that can be used to monitor and map dose received by the phantom in one, several, or numerous locations within the phantom.

In a more complex deployment, a continually-varying, spatially-dependent, density-equivalent material is fabricated using the 3D printing process to directly mimic the relevant portion of the computer-generated 3D model created using the process described below and on FIG. 2. This technique creates continually-varying material compositions (to mimic the tissue/bone density and energy-dependent effective Z numbers of that location) as a function of space and time that allows for subtle changes in density and effective radiation attenuation to be achieved in 3D and 4D and creates an even higher fidelity phantom than the other 3D printing technique described above.

To accomplish this, the spatially-dependent 3D matrix of HUs generated by the specially-defined registration process is increased in size to accommodate subsampling and the creation of a higher subsampled matrix of data. This HU matrix is then converted to a 3D model (or a series of 3D models that form a 4D model of the patient's medical image). As above, this model is tessellated (conversion from 3D cubic format to a nodal mesh of triangles) in software to smooth out the non-uniformities in the mesh.

This tessellated surface is then further smoothed to minimize the angular variation as a function of position of the planar surfaces with their adjoining surfaces until a smoothed model is then obtained from the pixelated rectangular data obtained from the medical image. The 3D model and the density-value matrix data is then converted into a computer language capable of operating the 3D printer described below. Unlike above, the phantom is created by the 3D printer using materials and fabrication techniques that allows for continuously variable material densities that directly mimic the density of the patient's tissues and bones. This is achieved through blending of various polymers, plastics, components, and fabrication techniques described below. As part of the fabrication process, miniature radiation dosimeters can be printed using tissue- and/or bone equivalent materials to provide a non-field perturbing radiation detection and measuring capability that can be used to monitor and map dose received by the phantom in one, several, or numerous locations within the phantom.

### 3D Printer Design

To fabricate multi-material, patient-specific, multi-dimensional anthropomorphic phantoms, a specially-designed 3D printer technology is required. The 3D printer is designed around three key considerations: size and control, extruder design, and temperature control.

### Size and Control

The 3D printer must be large enough to facilitate printing major body parts. This requires build volumes up to 250 cm×250 cm×250 cm. In this design, the print head component movements facilitate printing, while the phantom remains stationary during printing as opposed to moving the build table or build volume. For the required degree of precision, the print heads are moved by precision micro-stepping stepper motors driving gear-reduced ball and lead screws. This minimizes the backlash in the system and improves spatial resolution of the system.

### Extruder Design

The extruder system provides for co-extrusion, multi-material extrusion, and custom blending of polymers in real-time during the fabrication process. This is accomplished through predictive blending protocols for the polymers coupled with a mixing vessel and feed equipment to provide the needed plastic formulation to the extruder. A standard single, dual, or three screw extrusion systems are used to blend the needed formulation through a hot extrusion die that produces a small-diameter filament of the correct formulation. The formulation can be either tissue- or bone-equivalent with separate extruders for tissue and bone materials or by using one extruder capable of handling both tissue and bone formulations. Feedstock filament or pellet material can be used to feed into this extruder for FDM printing. This feedstock filament is then fed into the printing extruder and heated over its melt point. Using standard FDM techniques, the printer then prints the 3D object.

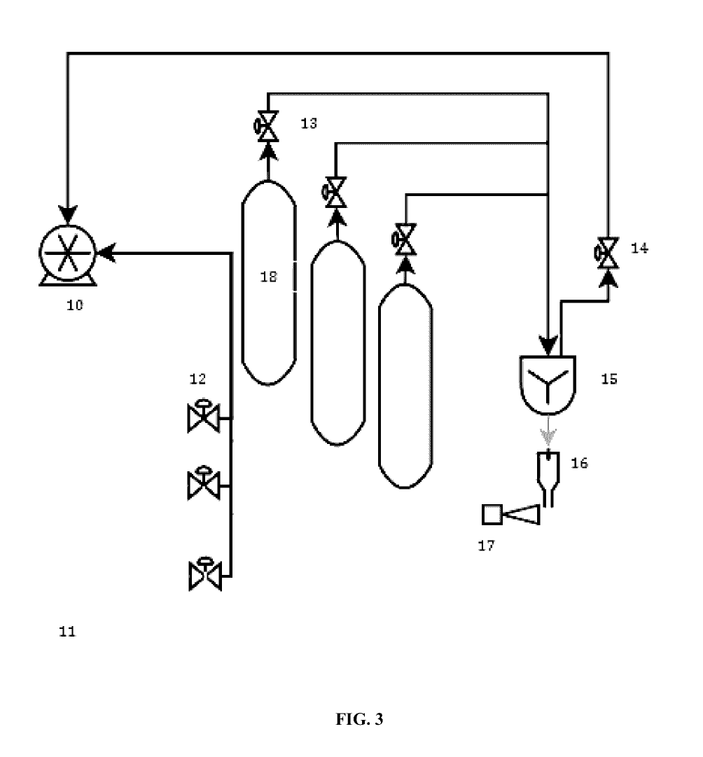

As depicted in FIG. 3, an exemplary extruder design is for multi-part, liquid polymer printing. Description of drawing. The extruder design in FIG. 3 consists of a vacuum pump 10 to provide negative pressure and suction needed for fabrication, a compressed air tank with air compressor 11, regulating valves 12 for providing pressure to solution tanks to push polymer components, metering valves 13 for binary, ternary, or others prior to mixing, a vacuum valve 14 on a mixing/extruder head for retracting flow between layers, a mixing and homogenizing system 15, and an external primary extruder head and hotend assembly 16. The regulating valves 12 can be a regulating valve, mass flow controller, or the like.

Many times, the polymer or plastic formulation cannot be made into a filament or pellets for extruding. To handle this case, tanks 18 of the precursor materials are kept in central location outside the printing bed. The tanks feed either by pressure or by mechanical metering into blending tank where two or more materials are blended together. This is achieved through a central, fixed or moving agitator which blends the chemical components together prior to being forced through the extruder nozzle. Surrounding the extruder nozzle are both heating and cooling devices 17 to aid in the removal of heat from this exothermic chemical process and maintaining the proper temperature given variations in temperature due to the chemical mixing and extrusion processes. Proper formulation is also achieved through precision metering, which is achieved through compressed gas insertion into the tank system or through a suitable pump, screw, or piston injection system. Retraction is controlled through precision gas removal up to vacuum levels.

UV laser light or UV light from LEDs, arc lamps, or other suitable source is directed onto the polymer stream exiting the nozzle to rapidly speed up curing and reduce the set time of the polymer. Preferably a mechanism capable of varying print-material composition in real time through filament mixing or incorporating polymer additives to the filament stream is provided. With this system, the composition of the polymers can be varied as needed to create the polymers with tissue equivalence from the radiation standpoint, e.g., air, bone, soft tissue, physical hardness (Shore Hardness number for use in time-dependent variations), or using transparent formulations for use in the direct-reading phantom described above.

Although the present invention has been described in considerable detail with reference to certain preferred versions thereof, other versions are possible. Therefore, the spirit and scope of the appended claims should not be limited to the description of the preferred versions contained herein. All features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

I claim:

1. A phantom wherein the phantom is constructed of a material having radiation attenuating properties mimicking human tissue.

2. The phantom of claim 1 wherein the material is a bone surrogate with a Hounsfield Unit (HU) value between 100 and 800.

3. The phantom of claim 1 wherein the material is a tissue surrogate with a HU between −500 and 100.

4. The phantom of claim 1 wherein the material is designed to mimic time-dependent physical and deformable characteristics of a patient resulting from precise control over Shore Hardness number which is controlled through polymer selection, degree and technique of cross linking, and selection of elemental species incorporated into a plastic.

5. The phantom of claim 1 wherein a time-dimensionally dependence for the phantom is achieved through attachment of the phantom to a mechanical means to modify the phantom.

6. A method of modifying a phantom wherein a deformable phantom is created by casting direct-reading radio-chromic/photochromic polymers, a resin base, and hardeners into a transparent matrix.

7. The method of claim 6 wherein the deformable phantom designed to simulate a digestive tract is manipulated with surrogate materials that mimic real patient conditions.

8. The method of claim 6 wherein the deformable phantom designed to simulate respiratory and pulmonary systems is printed as empty voids which are then inflated using an external, high-pressure, controlled and regulated system to mimic a patient's real life respiration.

9. The method of claim 6 wherein the deformable phantom designed to simulate cardiac system has fluidic pressure regulated with a suitable pump and a patient heart is mimicked through an electromechanical system.

10. A method of creating a phantom comprising the steps of:
a) performing a conversion between medical imaging data and digital HU values using a specially-defined registration process, and
b) using said digital HU values to select position-specific material for construction of the phantom.

11. The method of claim 10 further comprising the step of adjusting the density of the phantom by doping with materials selected from a group consisting of calcium, strontium, silicon, aluminum, potassium, bone trace elements, metals in an oxide, and metal in a calcined form.

12. The method of claim 10 further comprising the step of constructing the phantom with materials selected from a group consisting of binary, ternary, and multi-part resins, vinyls, urethanes, polymers, and elastomers which are binary, ternary, or multi-part including a resin base and a hardener.

13. The method of claim 10 further comprising the step of extruding the phantom using a filament for Fused Deposition Modeling (FDM).

14. The method of claim 10 further comprising the step of adjusting density of the phantom by doping with materials selected from a group consisting of air, water, solvents, and ethylene.

15. The method of claim 10 wherein non-field perturbing radiation dosimeters measuring less than 0.1 cc are embedded in the phantom.

16. The method of claim 10 further comprising the step of mixing, printing, and curing the phantom in real time as a modification to FDM.

17. The method of claim 15 wherein the non-field perturbing radiation dosimeters is painted with a paint selected from a group consisting of colloidal metallic and metallic conductive paint.

18. The method of claim 15 wherein the non-field perturbing radiation dosimeters is direct wire-bonded during fabrication with one or more interconnect wires.

19. The method of claim 15 wherein the non-field perturbing radiation dosimeters is printed with one or more coaxial cable.

20. The method of claim 15 wherein the non-field perturbing radiation dosimeters are non-field perturbing gas-filled ionization chambers comprised of:
a) a chamber wall of the dosimeter and a center terminal constructed from tissue equivalent materials, and
b) a set of one or more coaxial cables.

21. The method of claim 20 wherein an interior wall of the non-field perturbing gas-filled ionization chamber is flash evaporated with a metallic material.

22. The method of claim 19 wherein one or more coaxial cables is connected to a capacitor and one or more dosimeters.

23. The method of claim 22 wherein one or more dosimeters comprise:
a) a semiconductor diode dosimeter, and
b) a solid-state material selected from a group consisting of crystalline, amorphous, and powdered material.

24. The method of claim 23 wherein one or more dosimeters comprise polymers cast into a transparent matrix to which chemicals selected from a group consisting of the following are added: diarylethenes, azobenzenes, phenoxynaphthacene quinone, and metal halides.

25. The method of claim 24 further comprising a step wherein the polymers transition back to transparent after a decay period.

26. The method of claim 24 further comprising a step wherein the polymers do not transition back to transparent.

27. The method of claim 24 further comprising a step wherein the polymers are read out utilizing an optical system selected from a group consisting of directed laser scanning and digital optical imaging.

28. The method of claim 24 further comprising a step wherein the polymers are read out in a medium selected from a group consisting of air and immersed in a fluid with an index of refraction matching the phantom.

29. A three-dimensional printer technology using Fused Deposition Modeling (FDM) comprising:
a) a print head component utilizing all ball screw and lead screw construction,
b) an internal or external primary extruder to produce feedstock for FDM printing,
c) at least one extrusion nozzle,
d) a tank for resin,
e) a tank for hardener,
f) one or more other tanks for polymer additives,
g) a mixing vessel to uniformly mix and homogenize the polymer additives prior to extrusion,
h) a mechanism capable of picking up and placing of a dosimeters during print,
i) a mechanism capable of varying print-material composition in real time, and
j) a set of heating and cooling devices surrounding the extrusion nozzle.

30. The three-dimensional printer technology of claim 29 which further has the ability to connect radiation dosimeters to readout electronics.

31. The three-dimensional printer technology of claim 29 further comprising an agitator which blends polymer components together prior to being forced through the extrusion nozzle.

32. The three-dimensional printer technology of claim 29 further comprising precision metering prior to extrusion.

33. The three-dimensional printer technology of claim 29 which further achieves proper formulation of the liquid polymer in advance of when the material is required by the printer.

34. The three-dimensional printer technology of claim 29 in which the extrusion nozzle is surrounded by both heating and cooling devices to aid maintaining the proper temperature.
